(19) United States
(12) Patent Application Publication
(10) Pub. No.: US 2025/0367250 A1
(43) Pub. Date: Dec. 4, 2025

(54) AQUEOUS SOLUTION COMPRISING A GLUTATHIONE SALT

(71) Applicant: RENOVION, INC., Chapel Hill, NC (US)

(72) Inventors: Edward J. DELANEY, Princeton, NJ (US); Carolyn DURHAM, Chapel Hill, NC (US); Daniel W. COPELAND, Chapel Hill, NC (US)

(21) Appl. No.: 19/299,047

(22) Filed: Aug. 13, 2025

Related U.S. Application Data
(63) Continuation of application No. 18/821,027, filed on Aug. 30, 2024, now Pat. No. 12,419,932.
Application No. 18/821,027 is a continuation of application No. 18/640,964, filed on Apr. 19, 2024, now Pat. No. 12,097,238.
Application No. 18/640,964 is a continuation of application No. PCT/US2023/010081, filed on Jan. 4, 2023.
(60) Provisional application No. 63/296,405, filed on Jan. 4, 2022.

(51) Int. Cl. A61K 38/06; A61K 9/08; A61K 31/375

(52) U.S. Cl. CPC A61K 38/063; A61K 9/08; A61K 31/375

(57) ABSTRACT

This disclosure provides pharmaceutical preparations, products, and methods relating to ARINA-1 for use in treating patients with chronic inflammatory lung diseases, such as lung transplant, cystic fibrosis (CF), non-CF bronchiectasis, chronic obstructive pulmonary disease (COPD, and other inflammatory lung diseases.

### CROSS-REFERENCE TO RELATED APPLICATIONS

This application is a continuation of international application PCT/US2023/010081 filed Jan. 4, 2023, which claims the benefit of U.S. Provisional Application Ser. No. 63/296,405 filed Jan. 4, 2022, which are incorporated herein by reference in their entirety.

### BACKGROUND

### Field of the Disclosure

This disclosure relates to the general fields of chemistry and pharmaceutical preparations. In some aspects, the disclosure relates to pharmaceutical preparations, e.g., containing glutathione, suitable for delivery to the lung and methods of using the same. In some aspects, this disclosure provides pharmaceutical preparations and products relating to ARINA-1 for use in treating patients with chronic inflammatory lung diseases characterized by excess mucus and inflammation, such as lung transplant, cystic fibrosis (CF), non-CF bronchiectasis, chronic obstructive pulmonary disease (COPD), and other inflammatory lung diseases.

### Background

Excess mucus and inflammation are hallmarks of chronic inflammatory lung diseases. Ion dysregulation contributes to excess mucus production and chronic, dysregulated inflammation, both of which result in lung damage and scarring that leads to lung failure. Ramsey et al., American Journal of Respiratory and Critical Care Medicine 201(6):661-670 (2020); Borok et al., Lancet 338:215-216 (1991); Behr et al., Transplantation 69:1856-1860 (2000). Glutathione is a critical antioxidant ion that contributes to the structural integrity of mucus (Meldrum et al., Scientific Reports 8(1): Article number 5802 (2018) https://doi.org/10.1038/s41598-018-24223-3) and regulates inflammatory responses. Elferink, Immunobiology 184(1):25-36 (1991). Chronic inflammation also leads to increased airway acidity that contributes to lung damage. Glutathione dysregulation and increased airway acidity directly contribute to hyperviscous mucus that is accompanied by delayed or absent mucociliary clearance and consequent obstruction of small and medium-sized airways. This results in a milieu that favors a feedback loop of bacterial colonization, chronic infection, and inflammation.

Current therapies for chronic inflammatory lung diseases include dornase alfa and hypertonic saline. Dornase alfa (Pulmozyme; Genentech Inc.), an inhaled recombinant human deoxyribonuclease that cleaves extracellular DNA accumulated in CF mucus, Yang et al., Cochrane Database Syst Rev. 4: CD001127 (2016). Hypertonic saline, which is believed to hydrate the mucus layer through osmotically driven fluid transfer. Donaldson et al., N Engl J Med. 354:241-250 (2006). However, improvements in lung function, exacerbations, and quality of life observed with these agents are modest and variable. Yang et al., Cochrane Database Syst Rev. 4: CD001127 (2016); Henke et al., Paediatr Respir Rev. 8:24-29 (2007).

ARINA-1 is a compounded pharmaceutical preparation used to treat CF and other chronic inflammatory lung diseases. ARINA-1 is prepared by dissolving bicarbonate, L-glutathione, and ascorbic acid in water. The aqueous solution thus obtained is nebulized prior to administration to the lung of the patient. See, e.g., Adewale et al., Am J Respir Cell Mol Biol. 63:362-373 (2020); U.S. Pat. Nos. 9,308,234 and 11,058,743; and US Patent Appl. Nos. 2019/0351005 and 2020/0397849. There exists a need in the art for stable pharmaceutical preparations and products for use in lung disease therapy.

### BRIEF SUMMARY

This disclosure provides stable pharmaceutical preparations and products relating to ARINA-1 for use in treating patients with chronic inflammatory lung diseases characterized by excess mucus and inflammation, such as lung transplant, cystic fibrosis (CF), non-CF bronchiectasis, chronic obstructive pulmonary disease (COPD), and other inflammatory lung diseases.

The compounding of ARINA-1 and related pharmaceutical preparations comprises dissolving a bicarbonate salt, e.g., sodium bicarbonate, L-glutathione, and ascorbic acid in water. Applicant has discovered that such aqueous compositions are surprisingly stable when prepared, packaged, and stored under an atmosphere of carbon dioxide.

In one aspect, the disclosure provides a preparation comprising an aqueous solution in a closed container with a headspace, wherein:
(i) the aqueous solution comprises a salt having Formula I:

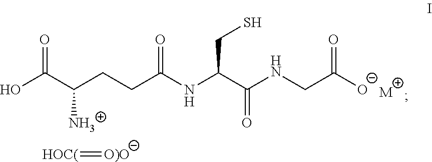

(ii) the atmosphere of the headspace comprises 90% or more carbon dioxide by volume; and
(iii) M⁺ is Na⁺, Li⁺, K⁺ or Cs⁺.

In another aspect, the disclosure provides a method of making a preparation comprising the aqueous solution comprising a salt having Formula I, the method comprising:
(i) dissolving L-glutathione, ascorbic acid, and M⁺HCO₃⁻, wherein M⁺ is Na⁺, Li⁺, K⁺ or Cs⁺, in water for injection under an atmosphere of carbon dioxide to give an aqueous solution;
(ii) transferring a portion of the aqueous solution to a container;
(iii) overlaying the aqueous solution with carbon dioxide; and
(iv) sealing the container with a stopper.

In another aspect, the disclosure provides an aqueous solution comprising a salt having Formula I prepared by dissolving L-glutathione, ascorbic acid, and M⁺HCO₃⁻, wherein M⁺ is Na⁺, Li⁺, K⁺ or Cs⁺, in water for injection under an atmosphere of carbon dioxide.

### BRIEF DESCRIPTION OF DRAWINGS

FIG. 1 is a line graph showing the pH versus time of an aqueous solution comprising sodium bicarbonate, L-glutathione, and ascorbic acid compounded under an atmosphere of nitrogen.

FIG. 2 is a line graph showing the pH versus time of an aqueous solution comprising sodium bicarbonate, L-glutathione, and ascorbic acid compounded under an atmosphere of carbon dioxide.

FIG. 3 is a partial NMR spectrum of an aqueous solution comprising sodium bicarbonate, L-glutathione, and ascorbic acid compounded under an atmosphere of carbon dioxide.

FIG. 4 is a flow diagram showing the compounding steps used to prepare ARINA-1 under an atmosphere of carbon dioxide.

### DETAILED DESCRIPTION

### I. Preparations of the Disclosure

In one aspect referred to as “Embodiment I,” the present disclosure provides a preparation comprising an aqueous solution in a closed container with a headspace, wherein:
(i) the aqueous solution comprises a salt having Formula I:

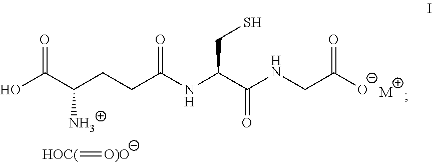

(ii) the atmosphere of the headspace comprises 90% or more carbon dioxide by volume; and
(iii) M⁺ is Na⁺, Li⁺, K⁺ or Cs⁺.

In another aspect, the aqueous solution of Embodiment I has a pH of 6.0±0.4 for 24 hours or more at about 5° C.

In another aspect, the aqueous solution of Embodiment I has a pH of 6.0±0.3 for 24 hours or more at about 5° C.

In another aspect, the aqueous solution of Embodiment I has a pH of 6.0±0.2 for 24 hours or more at about 5° C.

In another aspect, the aqueous solution of Embodiment I has a pH of 6.0±0.1 for 24 hours or more at about 5° C.

In another aspect, the aqueous solution of Embodiment I further comprises a salt having Formula II:

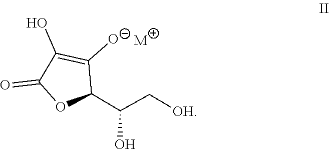

In another aspect, the aqueous solution of Embodiment I further comprises a salt having Formula III:

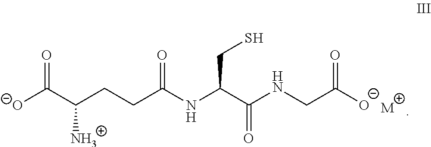

In another aspect, the atmosphere of the headspace of Embodiment I comprises 80% or more of carbon dioxide by volume.

In another aspect, the atmosphere of the headspace of Embodiment I comprises 85% or more of carbon dioxide by volume.

In another aspect, the atmosphere of the headspace of Embodiment I comprises 90% or more of carbon dioxide by volume.

In another aspect, the atmosphere of the headspace of Embodiment I comprises 95% or more of carbon dioxide by volume.

In another aspect, the aqueous solution of Embodiment I comprises about 10 wt % to about 20 wt % of the salt having Formula I.

In another aspect, the aqueous solution of Embodiment I comprises about 13 wt % to about 17 wt % of the salt having Formula I.

In another aspect, the aqueous solution of Embodiment I comprises about 14.7 wt % of the salt having Formula I.

In another aspect, the aqueous solution of Embodiment I comprises about 5 wt % to about 15 wt % of the salt having Formula II.

In another aspect, the aqueous solution of Embodiment I comprises about 7 wt % to about 11 wt % of the salt having Formula II.

In another aspect, the aqueous solution of Embodiment I comprises about 9.1 wt % of the salt having Formula II.

In another aspect, the aqueous solution of Embodiment I has a density of about 1.13 g/L.

In another aspect, the aqueous solution of Embodiment I is frozen.

In another aspect, M⁺ is Na⁺ in Embodiment I.

In another aspect, M⁺ is Li⁺ in Embodiment I.

In another aspect, M⁺ is K⁺ in Embodiment I.

In another aspect, M⁺ is Cs⁺ in Embodiment I.

In another embodiment, the preparation of Embodiment I is packaged as a single unit dose. In another aspect, the single unit dose is in a sealed vial.

In another aspect, the preparation of Embodiment I is marketed, distributed, or administered as part of a pharmaceutical product.

In some aspects, the preparation of Embodiment I can further comprise any one or more of the further aspects disclosed herein.

### II. Methods of Making a Preparation

In another aspect referred to as “Embodiment II,” the present disclosure provides a method of making the preparation of Embodiment I (or Embodiment I including one or more the further aspects disclosed above), the method comprising:
(i) dissolving L-glutathione, ascorbic acid, and M⁺HCO₃⁻, wherein M⁺ is Na⁺, Li⁺, K⁺ or Cs⁺, in water for injection under carbon dioxide to give an aqueous solution;
(ii) transferring a portion of the aqueous solution to a container;
(iii) overlaying the aqueous solution with carbon dioxide; and
(iv) sealing the container with a stopper.

In another embodiment, about 8 wt % to about 18 wt % of L-glutathione, about 3 wt % to about 13 wt % of ascorbic acid, and about 3 wt % to about 13 wt % of M⁺HCO₃⁻ is dissolved in about 62 wt % to about 82 wt % water for injection to give the aqueous solution of Embodiment II.

In another aspect, the aqueous solution of Embodiment II has a pH of 6.0±0.4 for 24 hours or more at about 5° C.

In another aspect, the aqueous solution of Embodiment II has a pH of 6.0±0.3 for 24 hours or more at about 5° C.

In another aspect, the aqueous solution of Embodiment II has a pH of 6.0±0.2 for 24 hours or more at about 5° C.

In another aspect, the aqueous solution of Embodiment II has a pH of 6.0±0.1 for 24 hours or more at about 5° C.

In another aspect, M⁺ is Na⁺, i.e., M⁺HCO₃⁻ is sodium bicarbonate, in Embodiment II.

In another aspect, M⁺ is Li⁺, i.e., M⁺HCO₃⁻ is lithium bicarbonate, in Embodiment II.

In another aspect, M⁺ is K⁺, i.e., M⁺HCO₃⁻ is potassium bicarbonate, in Embodiment II.

In another aspectt, M⁺ is Cs⁺, i.e., M⁺HCO₃⁻ is cesium bicarbonate, in Embodiment II.

In another aspect, about 11 wt % to about 15 wt % of L-glutathione, about 5 wt % to about 9 wt % of ascorbic acid, and about 5 wt % to about 9 wt % of sodium bicarbonate is dissolved in about 68 wt % to about 76 wt % water to give the aqueous solution of Embodiment II, wherein M⁺ is Na⁺.

In another aspect, about 13.0 wt % of L-glutathione, about 7.6 wt % of ascorbic acid, and about 7.3 wt % of sodium bicarbonate is dissolved in about 72.0 wt % water to give the aqueous solution of Embodiment II, wherein M⁺ is Na⁺.

In some aspects, the method of Embodiment II can further comprise any one or more of the further aspects disclosed herein.

### III. Product by Process

In another aspect referred to as “Embodiment III,” the present disclosure provides an aqueous solution comprising a salt having Formula I:

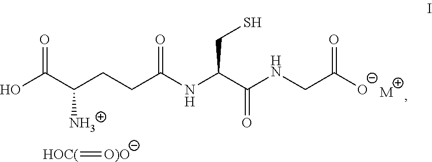

prepared by dissolving L-glutathione, ascorbic acid, and M⁺HCO₃⁻, wherein M⁺ is Na⁺, Li⁺, K⁺ or Cs⁺, in water for injection under an atmosphere of carbon dioxide.

In another aspect, about 8 wt % to about 18 wt % L-glutathione, about 3 wt % to about 13 wt % of ascorbic acid, and about 3 wt % to about 13 wt % M⁺HCO₃⁻; is dissolved in about 62 wt % to about 82 wt % water for injection to give the aqueous solution of Embodiment III.

In another aspect, the aqueous solution of Embodiment III has a pH of 6.0±0.4 for 24 hours or more at about 5° C.

In another aspect, the aqueous solution of Embodiment III has a pH of 6.0±0.3 for 24 hours or more at about 5° C.

In another aspect, the aqueous solution of Embodiment III has a pH of 6.0±0.2 for 24 hours or more at about 5° C.

In another aspect, the aqueous solution of Embodiment III has a pH of 6.0±0.1 for 24 hours or more at about 5° C.

In another aspect, the aqueous solution of Embodiment III further comprises a salt having Formula II:

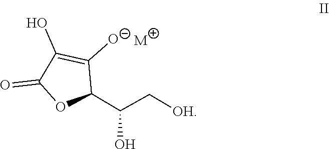

In another aspect, the aqueous solution of Embodiment III further comprises a salt having Formula III:

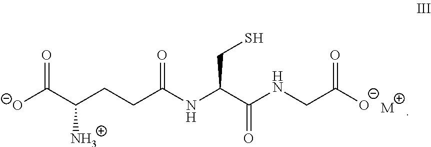

In another aspect, M⁺ is Na⁺, i.e., M⁺HCO₃⁻ is sodium bicarbonate, in Embodiment III.

In another aspect, M⁺ is Li⁺ , i.e., M⁺HCO₃⁻ is lithium bicarbonate, in Embodiment III.

In another aspect, M⁺ is K⁺, i.e., M⁺HCO₃⁻ is potassium bicarbonate, in Embodiment III.

In another aspect, M⁺ is Cs⁺, i.e., M⁺HCO₃⁻ is cesium bicarbonate, in Embodiment III.

In another aspect, about 11 wt % to about 15 wt % L-glutathione, about 5 wt % to about 9 wt % of ascorbic acid, and about 5 wt % to about 9 wt % sodium bicarbonate is dissolved in about 68 wt % to about 76 wt % water to give the aqueous solution in Embodiment III.

In another aspect, about 13.0 wt % L-glutathione, about 7.6 wt % ascorbic acid, and about 7.3 wt % sodium bicarbonate is dissolved in about 72.0 wt % water to give the aqueous solution in Embodiment III.

In some aspects, the aqueous solution of Embodiment III can further comprise any one or more of the further aspects disclosed herein.

### IV. Methods of Use

The preparations or aqueous solutions disclosed herein including those described above in Sections I-II (e.g., Embodiment I and III, and further aspects thereof) are useful for treating, reducing the symptoms of, or preventing a variety of diseases, conditions, or disorders. In particular, these preparations and aqueous solutions are useful in therapeutic methods of treating, reducing the symptoms of, or preventing a disease, condition, or disorder wherein the administration of L-glutathione, or a salt thereof, and/or ascorbic acid, or a salt thereof, provides a benefit.

In one aspect, the methods of treating or preventing a pulmonary or airway disorder in a subject in need thereof comprises administering, e.g., via inhalation, to the subject a therapeutically effective amount of a preparation described in Section I or an aqueous solution described in Section III.

In one aspect, the pulmonary or airway disorder is selected from the group consisting of chronic inflammatory lung disease, pulmonary fibrosis, pulmonary vasculitis, pulmonary sarcoidosis, an inflammation and/or infection associated with lung transplantation, acute or chronic lung acute or chronic rejection, chronic lung allograft dysfunction (CLAD, pulmonary artery hypertension, bronchitis, sinusitis, asthma, cystic fibrosis, a bacterial infection, a fungal infection, a parasite infection, a viral infection, chronic obstructive pulmonary disease (COPD), bronchiolitis obliterans syndrome (BOS), primary ciliary dyskinesia (PCD), alveolar protienosis, idiopathic pulmonary fibrosis, eosinophilic pneumonia, eosinophilic bronchitis, acute respiratory distress syndrome (ARDS), an inflammation and/or infection associated with mechanical ventilation, ventilator-associated pneumonia, complications associated with acute or chronic placement of a tracheostomy, an asbestos-related airway disorder or disease, a dust-related airway disorder or disease, silicosis, and a radiation or chemical agent-related airway disease or disorder, and any combination thereof.

In another aspect, the pulmonary or airway disorder is selected from the group consisting of chronic inflammatory lung disease, an inflammation and/or infection associated with lung transplantation, acute or chronic lung rejection, chronic lung allograft dysfunction (CLAD), asthma, cystic fibrosis, and chronic obstructive pulmonary disease (COPD), compliations associated with acute or chronic tracheostomy placement, and any combination thereof. In another embodiment, the method further comprises administering an additional therapeutic agent to the subject.

In another aspect, the pulmonary or airway disorder arises collaterally from radiation and/or chemotherapy in subjects having cancer.

In another aspect, the present disclosure provides methods of treating or preventing an infection in the airway of a subject in need thereof, the method comprising administering to the subject a therapeutically effective amount of a preparation described in Section I. In another embodiment, a preparation described in Section I is administered to the subject in combination with one or more antibiotics. The antibiotics can be administered locally to the lungs and/or systemically.

In another aspect, the present disclosure provides methods of treating or preventing inflammation in the airway of a subject in need thereof, the method comprising administering to the subject a preparation described in Section I.

In another aspect, the present disclosure provides methods of treating or preventing a disease or disorder in mucosal tissue in a subject in need thereof, the method comprising administering to the subject a therapeutically effective amount of a preparation described in Section I. Non-limiting examples of mucosal tissue include the mouth, nose, eye, ear, upper respiratory tract, lower respiratory tract, gastrointestinal tract, vagina, rectum and urethra.

In another aspect, the present disclosure provides methods of treating or preventing a disease or disorder associated with mucosal membranes, in a subject in need thereof, the method comprising administering to the subject a therapeutically amount of a preparation described in Section I to the appropriate mucosal membranes. In one embodiment, the mucosal membranes are the lungs, such the mid-bronchioles or the deep lung (alveolar region), and in other embodiments, the mucosal membranes are one or more of the eyes, mouth, nose, rectum, urogenital tract, and vagina.

In another aspect, a preparation described in Section I is used to treat or prevent bronchiolitis obliterans and military-related lung damage, i.e., lung damage of military personnel who have damaged airways secondary to unknown exposures.

The therapeutic methods of this disclosure comprise administering a therapeutically effective amount of a preparation described in Section I to a subject in need thereof, e.g., a human patient. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

In some aspect, a preparation described in Section I is nebulized prior to administration to a subject.

### V. Definitions

The term “ARINA-1” refers to the pharmaceutical preparation obtained by combining L-glutathione, ascorbic acid, and sodium bicarbonate in water to give the aqueous solution. The chemical entities present in ARINA-1 when prepared and maintained under an atmosphere of CO2 may include sodium L-ascorbate, L-glutathione sodium/bicarbonate salt, and L-glutathione sodium as shown in Scheme 1 .

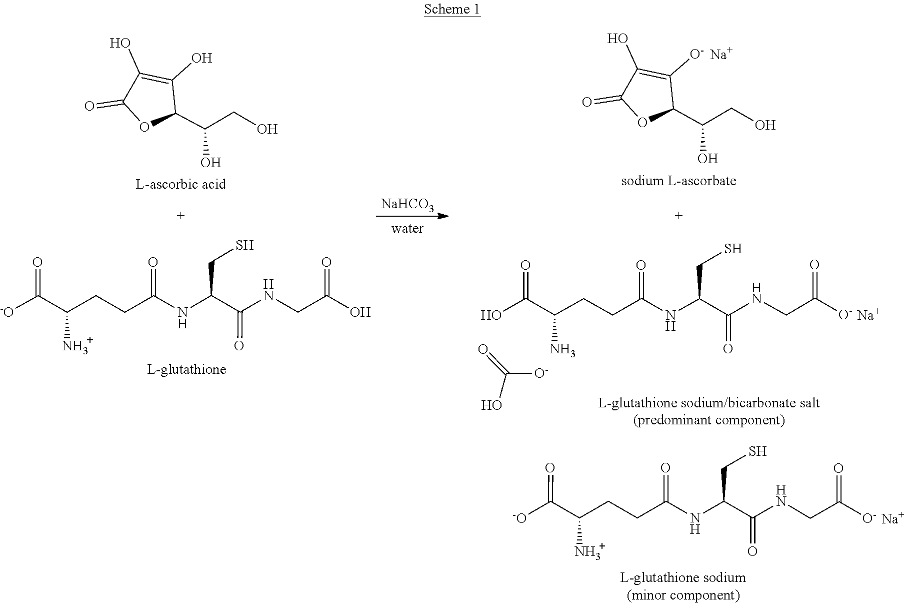

The stoichiometry of the components of ARINA-1 is shown in Table 1.

TABLE 1

| Formulation | Mol. Wt. | amount (g) | Moles | Rel. Equiv. |
|---|---|---|---|---|
| L-glutathione reduced | 307.32 | 150.0 | 0.488 | 1.00 |
| L-ascorbic acid | 176.12 | 88.0 | 0.500 | 1.02 |
| sodium bicarbonate | 84.01 | 84.0 | 1.000 | 2.05 |
| water | | 829.0 | | |

The term “pharmaceutical product” refers to a product suitable for human use. In some aspects, the pharmaceutical product is suitable for human use and is subject to regulation by the United States Food and Drug Administration (FDA) or a foreign equivalent of the FDA including, but not limited to, the European Medicines Agency (EMA), the National Medical Products Administration (NMPA) of China, and the Japanese Ministry of Health Labor and Welfare (MHLW).

The term “preparation” refers to one or more drugs intended for human use that is either (i) in its finished dosage form; or (ii) to be used for the preparation, formulation, and/or administration of the finished dosage form.

The term “container” refers to a pharmaceutically acceptable container comprising a chamber suitable to house an aqueous solution comprising one or more drug products. The “closed end” of the container refers to the end of the chamber having no opening. The term “open end” of the container refers to the end of the chamber opposite the closed end. Exemplary containers include, but are not limited to, vials, syringes, cartridges, bags, e.g., polypropylene and polyurethane bags, and ampoules. In one embodiment, the container is a vial.

The term “stopper” as used herein refers to any article capable of preventing an aqueous solution and a gas, e.g., carbon dioxide, from exiting the open end of a container.

The term “closed container” as used herein refers to a container affixed with a stopper.

The term “headspace” as used herein refers to the area within the chamber of a container between an aqueous solution and a stopper when the open end of the container is oriented away from the pull of gravity.

The term “atmosphere” as used herein refers to the layer of one or more gases in the headspace of a closed container. These gases include, but are not limited to oxygen, nitrogen, argon, and carbon dioxide. In one embodiment, the atmosphere in the pharmaceutical product of the present disclosure comprises 75% carbon dioxide or more, e.g., 80% carbon dioxide or more, 85% carbon dioxide or more, 90% carbon dioxide or more, 95% carbon dioxide or more, or 99% carbon dioxide or more, by volume.

The term “wt %” as used herein in connection with an aqueous solution refers to the mass of one component divided by the combined mass of all components (including, e.g., the water for injection) times 100. For example, the wt % of L-glutathione sodium/bicarbonate salt, i.e., a compound of Formula I, wherein M⁺ is Na⁺, in an aqueous solution comprising 161 g of L-glutathione sodium/bicarbonate salt, 99 g of sodium ascorbate, i.e., a compound of Formula II, wherein M⁺ is Na⁺, 1 gram of sodium bicarbonate, and 829 g of water is 14.8 wt % (161 g/1090 g=0.148×100=14.8 wt %). Likewise, the wt % of L-glutathione when 150 g of L-glutathione, 88 g of ascorbic acid, and 84 g of sodium bicarbonate is dissolved in 829 g of water to give an aqueous solution is 13.0 wt % (150 g/1151 g=0.130×100=13.0 wt %).

The terms “a” and “an” refer to one or more than one.

The term “about,” as used herein, includes the recited number ±10%. Thus, “about 10” means 9 to 11.

### EXAMPLES

### General Methods

### Materials

L-ascorbic acid (Lot #1200851004) and L-glutathione reduced (Lot #B200465) used in the 2-L scale experiment were sourced from CSPC Weisheng Pharmaceutical (Shijiazhuang) Co. and Shandong Jincheng Biopharmaceutical Co., respectively. L-ascorbic acid (Product #A92902) and L-glutathione reduced (Product #G4251) used in the 0.75-L scale experiment run involving carbon dioxide blanketing were purchased from Millipore Sigma Co. Sodium bicarbonate (Product #S6014) for all trials was purchased from Millipore Sigma Co. Deionized water was provided from a Millipore water system. All other raw materials and solvents used to analyze the product were purchased from commercial vendors and used as received.

### NMR Instrumentation

NMR was employed to assess the titer of L-ascorbic acid, L-glutathione reduced, L-glutathione disulfide and any unknown impurities in the demonstration runs. ¹H NMR spectra were obtained at 25° C. using a Bruker Avance III 500 MHz NMR system, equipped with a 5-mm, triple-resonance Cryoprobe. Deuterium oxide was used as the NMR solvent, and the resonance from calcium formate was used as a reference to establish chemical shift and integration values. NMR spectral data were processed using MNova software.

### Example 1 Compounding of ARINA-1 Under N₂

A 2-L scale preparation of ARINA-1, see Table 2, was carried out in a jacketed 4-neck glass reactor fitted with an overhead stirrer, a thermometer, a gas inlet adapter, a port for solids charging, and a second adapter to constrict the flow of nitrogen leaving the flask via a Claisen adapter. Nitrogen was charged to the flask via a flow meter capable of measuring flow between 0.05-0.5 L/min. Temperature control of the reaction was achieved by recirculating a water/glycol mixture from the vessel jacket into a Haake EZ-Cool 80 recirculating heater/chiller.

TABLE 2

| Formulation | MW | amount (g) | Moles | Rel. Equiv. |
|---|---|---|---|---|
| glutathione | 307.32 | 300.0 | 0.976 | 1.00 |
| Ascorbic acid | 176.12 | 176.0 | 0.999 | 1.02 |
| sodium bicarbonate | 84.01 | 168.0 | 2.000 | 2.05 |
| water | | 1600.0 | | |
| water rinse | | 58.0 | | |
| TOTAL MASS ADDED | | 2244.0 | | |
| O2 | 32 | 0.009 | 0.001125 | 0.12% |
| | 32 | 0.001 | 0.00003125 | 0.01% |

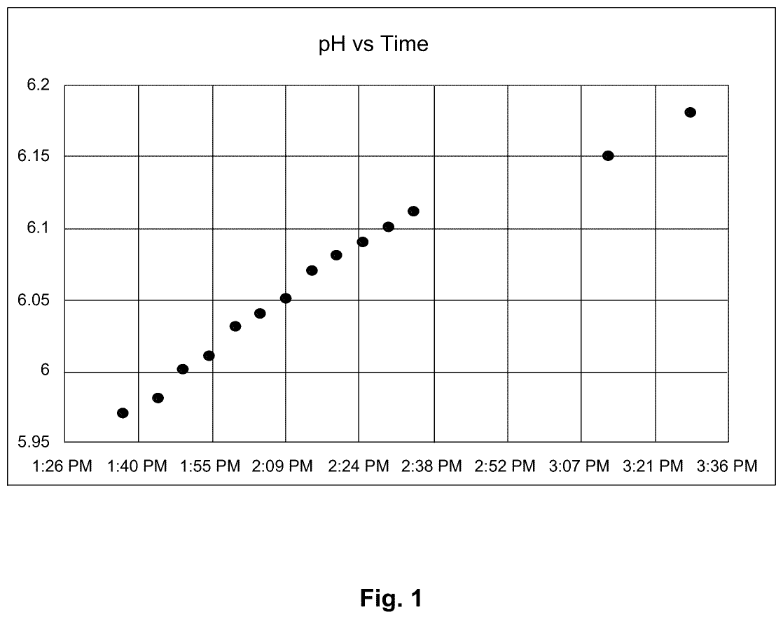

The pH was monitored at the end of L-glutathione neutralization, and an upward increase in pH was measured to be about +0.1 pH unit per hour at the temperatures (° C.) indicated in Table 3 and FIG. 1

TABLE 3

| | pH | temp |
|---|---|---|
| 1:38 PM | 5.97 | 18.4 |
| 1:45 PM | 5.98 | 16.5 |
| 1:50 PM | 6.00 | 14.7 |
| 1:55 PM | 6.01 | 12.9 |
| 2:00 PM | 6.03 | 11.4 |
| 2:05 PM | 6.04 | 10.0 |
| 2:10 PM | 6.05 | 8.7 |
| 2:15 PM | 6.07 | 7.7 |
| 2:20 PM | 6.08 | 6.7 |
| 2:25 PM | 6.09 | 5.9 |
| 2:30 PM | 6.10 | 5.0 |
| 2:35 PM | 6.11 | 4.0 |
| 2:40 PM | | |
| 3:13 PM | 6.15 | −1.7 |
| 3:29 PM | 6.18 | −2.3 |

### Example 2 Compounding of ARINA-1 Under CO₂

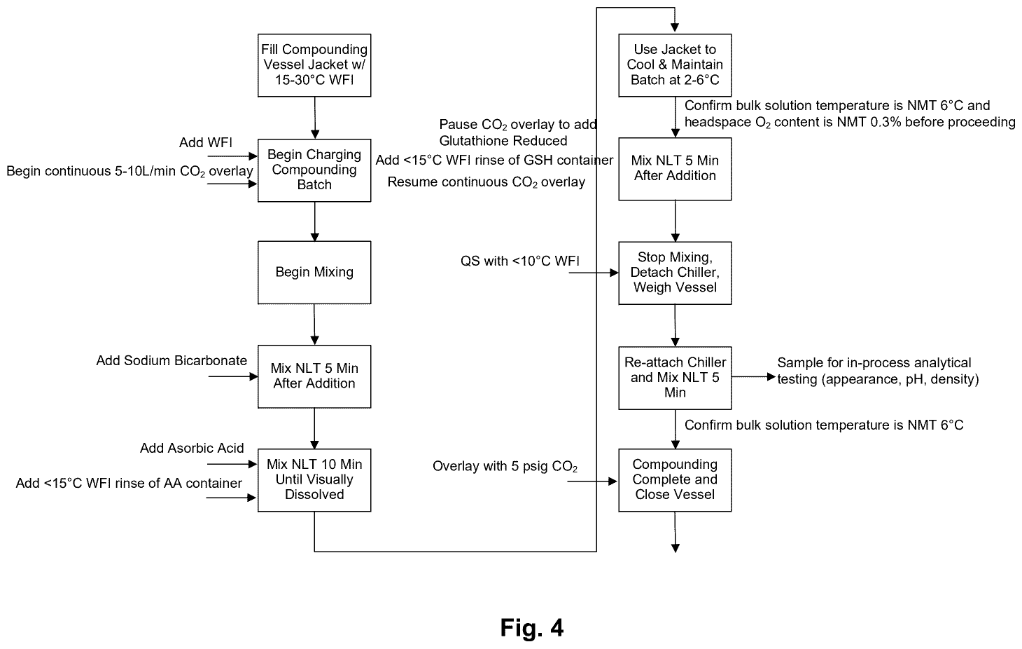

A 750-mL scale preparation of ARINA-1, see Table 4, was carried out in a 1-L jacketed 4-neck glass reactor fitted with an overhead stirrer, a thermometer, a gas inlet adapter, a port for solids charging. Carbon dioxide was charged to the reactor via a gas flow meter capable of measuring between 0.05-0.5 L/min. Temperature control of the reaction was achieved by recirculating a water/glycol mixture between the vessel jacket and a Haake EZ-Cool 80 recirculating heater/chiller. The compounding process steps are outlined in FIG. 4.

TABLE 4

| Formulation | MW | amount (g) | Moles | Rel. Equiv. |
|---|---|---|---|---|
| glutathione | 307.32 | 112.5 | 0.366 | 1.00 |
| Ascorbic acid | 176.12 | 66.0 | 0.375 | 1.02 |
| sodium bicarbonate | 84.01 | 63.0 | 0.750 | 2.05 |
| water | | 600.0 | | |
| water #2 | | 21.75 | | |
| TOTAL MASS ADDED | | 841.5 | | |

### Process Description

1. Added 600 g of deionized water to the reactor. This amount of water, along with the rinse introduced in step 6, gave a product density of 1.12 to 1.13 g/mL. Began agitation by setting the overhead stirrer rate to about 90-100 rpm. The temperature of the water in the demonstration trial was 21° C.
2. Added 63.0 g of sodium bicarbonate and continued agitation for about 10 minutes to allow most of the sodium bicarbonate to dissolve.
3. Displaced air in the headspace of the flask completely using gaseous carbon dioxide from a cylinder source fitted with an appropriate gas regulator.
4. Charged 66.0 g of L-ascorbic acid to the flask at a rate that ensured controlled evolution of carbon dioxide gas. Once the solid addition was done, the reaction was complete within 2-3 minutes resulting in a clear solution, after which time step 5 may commence.
5. Charged 112.5 g of L-glutathione reduced to the flask at a rate that ensured controlled evolution of carbon dioxide gas. Reaction was complete within 4-5 minutes following the last solid addition, resulting in a clear solution.
6. Measured out 21.75 g of deionized water, and used it to rinse any solids remaining in the solids addition funnel and/or the upper vessel wall that may have adhered solids present. Increased the rate of agitation dramatically and held for 10-15 seconds to splash any solid or liquid clinging to the vessel wall in order to achieve a completely homogeneous mixture.
7. Returned to a slower rate of agitation (but maintained an agitation rate sufficient to promote heat transfer) and immediately cooled the solution to between 2 to 6° C.

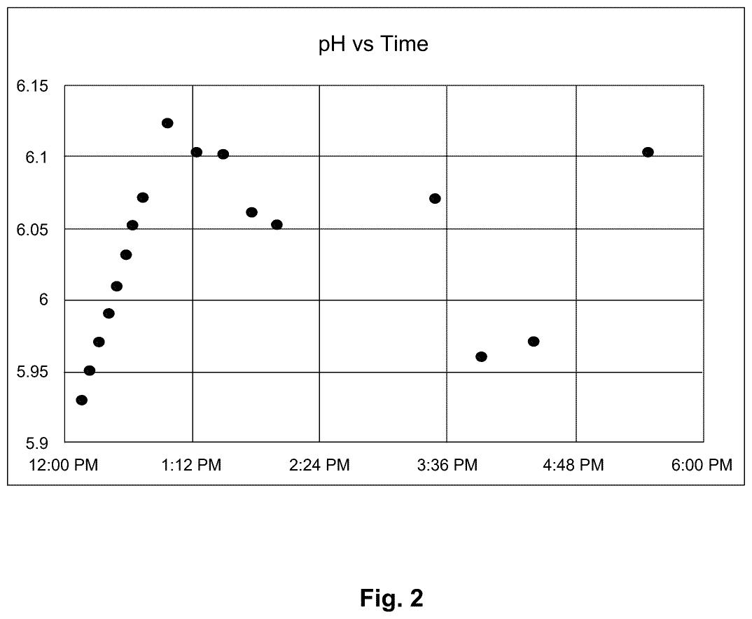

The pH was monitored at the end of L-glutathione neutralization at the temperatures (° C.) indicated in Table 5 and FIG. 2.

TABLE 5

| | pH | temp | |
|---|---|---|---|
| 12:10 PM | 5.93 | 14.5 | |
| 12:15 PM | 5.95 | 13.3 | |
| 12:20 PM | 5.97 | 11.5 | still slowly bubbling |
| 12:25 PM | 5.99 | 9.6 | |
| 12:30 PM | 6.01 | 8.0 | bubbling barely evident |
| 12:35 PM | 6.03 | 6.7 | |
| 12:40 PM | 6.05 | 5.4 | no bubbling evident |
| 12:45 PM | 6.07 | 3.5 | |
| 1:00 PM | 6.12 | 0.0 | |
| 1:15 PM | 6.10 | 0.0 | |
| 1:30 PM | 6.10 | 0.0 | bath temp increased to 5 deg |
| 1:45 PM | 6.06 | 4.1 | |
| 2:00 PM | 6.05 | 3.3 | |
| 3:30 PM | 6.07 | 0.0 | bath set to 15 deg |
| 3:55 PM | 5.96 | 13.8 | slow bubbling returned |
| 4:25 PM | 5.97 | 15.0 | bath temp returned to 1 deg C. |
| 5:30 PM | 6.10 | 1.0 | |
| 10:00 AM | 6.00 | 4.0 | |
| next day | | | |

The issue with continuous sparge or sweep of nitrogen in EXAMPLE 1 is that the nitrogen flow strips residual CO₂ out of the system, which in turn drives the decomposition of carbonic acid formed by the neutralization reactions. This increases pH above the target range of pH 5.8-6.4. In an experiment using a slow nitrogen sweep, the pH of the compounded product rose from 6.0 to 7.3 after approximately 18 hours of post-reaction inerting (data not shown).

Henry's gas law states that the amount of dissolved gas in a liquid is proportional to its partial pressure above the liquid. The partial pressure of CO₂ in the headspace above the product solution is kept close to zero under the condition of a slow, continuous nitrogen sweep. If, however, gaseous CO₂ is used to inert the headspace, the pressure of CO₂ becomes 1 atmosphere, which should provide a driving force to reverse CO₂ loss and establish an equilibrium that stabilizes pH. This principle was demonstrated in this example.

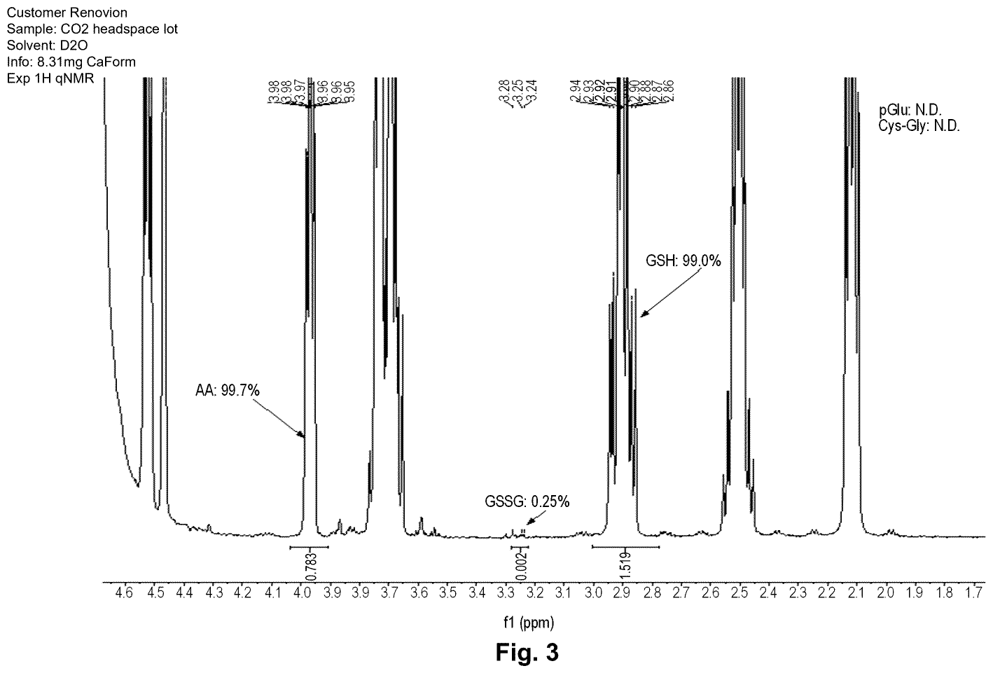

L-glutathione reduced is an acid that is being neutralized by sodium bicarbonate, and that gaseous CO₂ evolution should not be expected to stop until an equilibrium is established between the dissolved CO₂ in solution and CO₂ in the headspace. As in prior runs under, e.g., nitrogen, the pH rose following solid dissolution from about 5.95 to 6.10 while gas evolution was still evident as the solution was being cooled to 2-6° C. Equilibrium was established once the target pH range of 6.0-6.1 was reached. Raising the temperature and lowering it over the next 4 hours of the experiment did not have a significant effect on pH. After the solution was maintained under a slight positive pressure of CO₂ over 20+ hours a pH of 6.0 was again obtained. The solution was sampled for NMR analysis and gave assay values of 99.7 and 99.0% for L-ascorbic acid and L-glutathione reduced, respectively. See FIG. 3. The disulfide level was 0.25%, and no unexpected resonances were found that would indicate unaccounted degradants at or above 0.2% level. The use of a CO₂ blanket thus unexpectedly stabilizes the pH at its target range, as needed to permit consistency of product pH (and CO₂ content) throughout the vial filling cycle, while also excluding oxygen and controlling disulfide formation.

All inputs to this example were carefully measured by weight, permitting an analysis of the actual product CO₂ content that corresponds to pH 6.1. As shown in the stoichiometry calculations below for a 1-L preparation of ARINA-1, if all CO₂ were to have been expelled the product should be expected to weigh 1107.6 g, while if all CO₂ were unexpectedly retained in solution the product should weigh 1151.0 g. A 500-mL portion of preparation of this example was weighed and found to be 1125.8 g/L.

| Formulation | Mol. Wt. | amount (g) | Moles | Rel. Equiv. |
|---|---|---|---|---|
| glutathione | 307.32 | 150.0 | 0.488 | 1.00 |
| Ascorbic acid | 176.12 | 88.0 | 0.500 | 1.02 |
| sodium bicarbonate | 84.01 | 84.0 | 1.000 | 2.05 |
| water | | 800.0 | | |
| water #2 (rinse) | | 29 | | |

↓ (post-neutralization)

| | | | | |
|---|---|---|---|---|
| Na glutathionate generated | 329.3 | 160.7 | 0.488 | 1.00 |
| Na ascorbate generated | 198.1 | 99.1 | 0.500 | 1.02 |
| sodium bicarbonate remaining | 84.01 | 1.0 | 0.012 | 0.02 |
| water | | 800.0 | | |
| water #2 (rinse) | | 29 | | |
| water generated from bicarbonate | 18.02 | 17.8 | 0.988 | |
| CO2 potentially lost | 44 | 43.5 | 0.988 | |
| TOTAL MASS-if all CO2 lost | | 1107.6 | | |
| TOTAL MASS-if no CO2 lost | | 1151.0 | | |
| TOTAL MASS-actually measured | | 1125.8 | | |
| Difference (as H2CO3) | 62.02 | 18.2 | 0.29 | |

This corresponds to a carbonic acid content of 18.2 g/L (0.29 moles), or about 30% of a molar equivalent to the two APIs combined. Given that the solubility of CO₂ in water under 1 atmosphere of CO₂ at 020 C. is 0.034 g/L, it may be inferred that the solutes present help stabilize carbonic acid in an unexpected manner. The L-glutathione reduced sodium salt is most likely responsible for this stabilization, as it is capable of forming a carbonic acid complex:

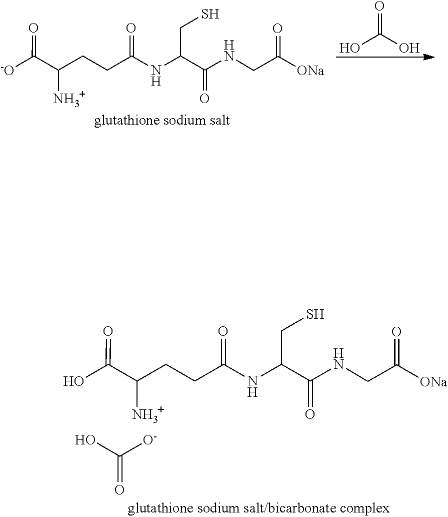

Having now fully described the compounds, compositions, methods, preparations, and products herein, it will be understood by those of skill in the art that the same can be performed within a wide and equivalent range of conditions, formulations, and other parameters without affecting the scope of the compounds, compositions, methods, preparations, and products provided herein or any embodiment thereof. All patents, patent applications, and publications cited herein are fully incorporated by reference herein in their entirety.

1-20. (canceled)

21. A aqueous solution comprising a salt having Formula I: 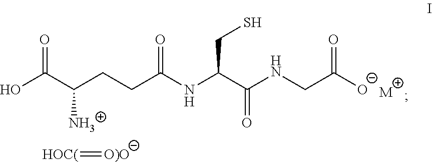
prepared by:
dissolving L-glutathione, ascorbic acid, and sodium bicarbonate in water for injection under an atmosphere of carbon dioxide, wherein:
M⁺ is Na⁺; and
about 8 wt % to about 18 wt % L-glutathione, about 3 wt % to about 13 wt % of ascorbic acid, and about 3 wt % to about 13 wt % sodium bicarbonate is dissolved in about 62 wt % to about 82 wt % water to give the aqueous solution.

22. The aqueous solution of claim 21, wherein the aqueous solution has a pH of 6.0±0.4 for 24 hours or more at about 5° C.

23. The aqueous solution of claim 22, wherein the aqueous solution has a pH of 6.0±0.1 for 24 hours or more at about 5° C.

24. The aqueous solution of claim 21, wherein the aqueous solution further comprises a salt having Formula II: 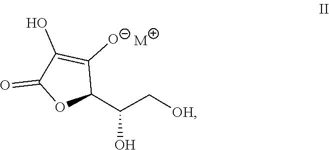
wherein M⁺ is Na⁺.

25. The aqueous solution of claim 21, wherein the aqueous solution further comprises a salt having Formula III: 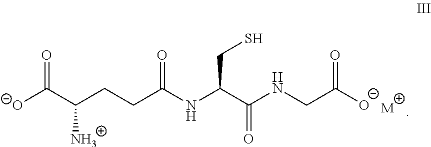
wherein M⁺ is Na⁺.

26. The aqueous solution of claim 21, wherein about 11 wt % to about 15 wt % L-glutathione, about 5 wt % to about 9 wt % of ascorbic acid, and about 5 wt % to about 9 wt % sodium bicarbonate is dissolved in about 68 wt % to about 76 wt % water to give the aqueous solution.

27. The aqueous solution of claim 26, wherein about 13.0 wt % L-glutathione, about 7.6 wt % ascorbic acid, and about 7.3 wt % sodium bicarbonate is dissolved in about 72.0 wt % water to give the aqueous solution.
